(19) United States
(12) Patent Application Publication
(10) Pub. No.: US 2006/0286600 A1
(43) Pub. Date: Dec. 21, 2006

(54) High throughput one-hybrid system

(76) Inventors: Albertha Walhout, Princeton, MA (US); Marc Vidal, Stoughton, MA (US); Bart Deplancke, Worcesta, MA (US)

Correspondence Address: FISH & RICHARDSON PC, P.O. BOX 1022, MINNEAPOLIS, MN, 55440-1022, US

(21) Appl. No.: 10/561,762

(22) PCT Filed: Jun. 30, 2004
(86) PCT No.: PCT/US04/20895

§ 371 Date: Jun. 7, 2006

Related U.S. Application Data
(60) Provisional application No. 60/484,193, filed on Jun. 30, 2003.

(51) Int. Cl. G01N 33/53; C12N 15/74; C12N 1/18

(52) U.S. Cl. 435/7.1; 435/254.2; 435/483

(57) ABSTRACT

The invention relates to a new high throughput yeast one-hybrid screening system.

### CLAIM OF PRIORITY

This application claims the benefit of priority under 35 USC §119(e) to U.S. Provisional Patent Application Ser. No. 60/484,193, filed on Jun. 30, 2003, the entire contents of which are incorporated by reference herein.

### TECHNICAL FIELD

This invention relates to a new one-hybrid system and uses of that system in high throughput screening of DNA sequences to identify and study DNA binding proteins.

### BACKGROUND

One-hybrid systems, such as yeast one-hybrid systems, are genetic assays that can be used to isolate novel genes encoding proteins that bind to a “bait element,” e.g., a cis-acting regulatory element or any other short DNA sequence (Wei et al., Mol. Cell. Biol., 19:1271-1278, 1999). With such one-hybrid assays, DNA-protein interactions are detected while proteins are in their native configurations, and the genes encoding these binding proteins are available immediately after a library screening.

Yeast one-hybrid systems have several uses. As noted above, they can be used to identify proteins that bind to a DNA element of interest. In addition to their use to identify transcriptional activators and repressors, the one-hybrid system can also be used to identify co-factors that repress the activity of an activator. Finally, the one-hybrid systems can be used to identify proteins that do not necessarily play a role in transcription regulation, but interact with certain DNA elements and may function in replication or other chromosome-related processes. Similar to the two-hybrid system, the one-hybrid system is based on the activation of reporter gene expression by a hybrid protein in which an open reading frame of interest is fused to the activation domain of a transcription factor. When the fusion protein binds to a promoter of interest through its cognate DNA binding domain, reporter gene expression is activated by the activation domain part of the fusion protein and can be efficiently selected for in yeast.

### SUMMARY

The invention relates, at least in part, to an improved high-throughput version of a yeast one-hybrid system. The system facilitates high-throughput, one hybrid analysis by using lambda recombination sites for insertion of a DNA bait element rather than by traditional cloning with restriction enzymes. Also, only one copy of the DNA bait element is required in this new system, and the system can be used with substantially longer DNA sequences than possible in prior systems. Furthermore, these methods can be carried out in intact cells, and do not require reconstitution in vitro.

Since the advent of microarrays, vast amounts of gene expression data have been generated. However, these microarray data fail to reveal the transcription regulatory mechanisms that underlie differential gene expression, because the identity of the responsible transcription factors (TFs) often cannot be directly inferred from such datasets. Regulatory TPs activate or repress transcription of their target genes by binding to cis-regulatory elements that are frequently located in a gene's promoter. To understand the mechanisms underlying differential gene expression, it is necessary to identify physical interactions between regulatory TFs and their target genes. The methods described herein can be used to explore these interactions, to thereby identify new drug targets, and evaluate the effects of potential therapeutics on gene transcription.

In some aspects, the invention provides methods for identifying proteins that associate with, e.g., bind to, a bait element. The methods include obtaining a cell, e.g., a yeast cell, such as a YM4271 cell, or a mammalian cell whose genome contains one or more integrated bait-reporter constructs, wherein each of the one or more bait-reporter constructs includes (a) a single copy of a bait element flanked by lambda recombination sites, wherein the bait element contains at least about 250 base pairs, e.g., about 500, 750, 1000, 1250, 1500, 2000, or 2500 base pairs, and (b) a reporter gene, e.g., HIS3 and/or LacZ; transforming the cell with an expression vector encoding a fusion protein comprising an activation domain; and assessing activation of the reporter gene. Activation of the reporter gene indicates that the activation domain fusion protein has associated with the bait element. In some embodiments, activation is assessed by plating the yeast cell on nutrient deficient plates under growth conditions, and observing any growth of yeast cells. In some embodiments, activation is assessed by determining the color of the yeast cells.

In some embodiments, two reporter genes are used, and the reporter gene can be a fluorescent gene, e.g., luciferase, green fluorescent protein, yellow fluorescent protein, red fluorescent protein, blue fluorescent protein, or a fluorescent variant of any of these proteins.

In some embodiments, the fusion protein is an activation domain-transcription factor fusion protein.

In some embodiments, the methods include selecting a cell in which a reporter gene is activated; and isolating the cDNA encoding an activation domain fusion protein that is associating with the bait element from the cell. In some embodiments, the methods include determining the sequence of the isolated cDNA.

In some embodiments, the methods include contacting the cell with a test compound, and evaluating the effect of the test compound on the association (e.g., binding) between the bait element and the activation domain fusion protein.

The new system can be used to screen any DNA sequences (“bait elements”), including promoters from species from C. elegans to Homo sapiens, to identify associated DNA-binding proteins and transcription factors. In addition, the system can be used to screen other DNA sequences from human genomic DNA or DNA from other organisms.

As used herein a “bait element” is a sequence of DNA that associates with, e.g., is bound by, a transcription factor. Typically, the bait element includes all or part of a promoter sequence. However, the bait element can also comprise DNA sequences that lie further upstream (e.g., enhancers) or downstream (e.g., introns) of the transcription start site. In some embodiments, the bait element is at least about 250, 500, 750, 1000, 1500, 2000 or 2500 bases long. The ability to use longer bait elements allows the identification of longer and more complicated binding sites, and the identification of more distant elements that affect transcription. The promoter can include intergenic sequences upstream of the predicted start of each open reading frame. In some embodiments, the promoter has a maximal length of about 2.5 kilobases.

As used herein a “reporter gene” is any gene used to indicate that transcription is occurring. For example, a gene essential to survival of the cell can be a reporter gene, because survival of the cell indicates that transcription of the gene is occurring.

As used herein a “bait-reporter construct” is an oligonucleotide that includes a bait element together with a reporter gene where the bait element is upstream of the reporter gene. If a gene name precedes the term “bait-reporter construct,” the gene name refers to the type of reporter used in the bait-reporter construct.

As used herein, an “activation domain” is a domain of a transcription factor that activates transcription of a target gene.

As used herein, a “fusion protein comprising an activation domain” is a fusion protein that includes an activation domain of a transcription factor as defined above and a potential, suspected or known DNA binding protein, e.g., a transcription factor.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representation of a standard yeast one-hybrid system. A sequence of multiple tandem copies of a known bait element is inserted upstream of a reporter gene and integrated into the yeast genome to make a new reporter strain. The yeast strain is transformed with an activation domain fusion library to screen for DNA-binding proteins that interact with the bait DNA sequence. Binding of an activation domain/DNA-binding protein (AD/DNA-BP) hybrid protein to the bait sequence results in activation of reporter gene transcription.

FIG. 2 is a schematic representation of a standard GATEWAY® PCR cloning reaction (Invitrogen, Carlsbad, Calif.).

FIG. 3 is a schematic representation of a standard GATEWAY® LR reaction (Invitrogen).

FIG. 4A is a schematic representation of a non-GATEWAY vector, including an Ampicillin resistance gene (Amp^{r}), a p53 binding sequence (p53BS), and a HIS3 reporter gene.

FIG. 4B is a set of three panel representing control colonies (leftmost three) and colonies transformed with the vector illustrated in FIG. 4A, grown in 0, 20, or 40 mM 3-aminotriazole (3AT).

FIG. 4C is a schematic representation of a GATEWAY style vector, including an Ampicillin resistance gene (Amp^{r}), a p53 binding sequence (p53BS), and a HIS3 reporter gene, as well as three modified attb sites (B4, B2, and B1).

FIG. 4D is a set of three panel representing control colonies (leftmost three) and colonies transformed with the vector illustrated in FIG. 4C, grown in 0, 20, or 40 MM 3AT.

FIGS. 5A-5E are representations of colony growth in yeast transformed with Pfog-3 (top 2 rows), Phlh-8 (middle two rows), or p53BS (bottom rows), showing the results of a self-activation test under permissive (FIG. 5A) and progressively more stringent selective conditions (5B, 20 mM 3-aminotriazole (3AT); 5C, 40 mM 3AT; 5D, 60 mM 3AT; 5E, 80 mM 3AT).

FIG. 5F is a representation of colony growth in yeast, showing the results of a beta-galactosidase assay in yeast transformed with Pfog-3 (top 2 rows), Phlh-8 (middle two rows), or p53BS (bottom rows).

FIG. 5G is a representation of a yeast growth plates that shows the results of replica plating of yeast from the colonies labeled 1, 2, and 3 in FIG. 5B, on plates with 0 mM 3AT (left image) or 20 mM 3AT (right image).

FIGS. 6A to C are a series of representations of colony growth showing the interaction of various promoter::HIS3 constructs with regulatory transcription factors (“RTF”) known to bind to the specific promoters.

FIG. 7A-B are a series of representations of a beta galactosidase assay showing the interaction of various promoter::LacZ vectors with regulatory transcription factors (“CRTF”) known to bind to the specific promoters; darker colonies (blue in original) indicate activation of the LacZ reporter.

FIG. 8 is a bar graph showing the results of luciferase reporter expression in mouse 3T3 cells transfected with six various transcription factors and a control empty GST.

FIG. 9 is a schematic illustration of a method of making two vectors containing bait reporter constructs.

### DETAILED DESCRIPTION

The new yeast one-hybrid system described herein facilitates the high-throughput identification of binding proteins that associate with bait elements inserted into yeast one-hybrid vectors. The methods of making and using this system are described below. The new system can be used to screen genomic DNA, e.g., promoters, of any species, from C. elegans to Homo sapiens, and for any gene of interest, to identify associated DNA-binding proteins and transcription factors.

It is generally accepted that expression of certain genes in a mutated form is associated with certain diseases (e.g., expression of a mutated p53 is correlated with many cancers). Identifying the transcription factors that regulate the expression of these genes will be helpful in identifying viable drug targets. Also, identifying other promoters that are regulated by a certain transcription factor will help in identifying possible side effects in targeting that particular transcription factor. For example, identifying transcription factors that regulate p53 expression will help identify possible cancer drug targets, and identifying other genes that those p53 transcription factors regulate will help in identifying some of the possible side effects of targeting that transcription factor with an anti-cancer drug. A high-throughput system able to quickly identify transcription factor—promoter associations (e.g., binding) will speed up the process of identifying possible drug targets and identifying possible side effects of a drug targeting a particular transcription factor.

General Methodology

Standard one-hybrid assays generally require that (1) the sequence of the bait element must be known; (2) the sequence of the bait element must be short (e.g., less than about 30 bp); and (3) multiple copies, e.g., 4, 5, or more copies, of the bait element must be used to get a sufficient signal. (Ghosh et al., J. Mol. Biol. 234(3):610-9 (1993); Clonetech MATCHMAKER® One-hybrid System User Manual, Catalog #K1603-1 (1997)).

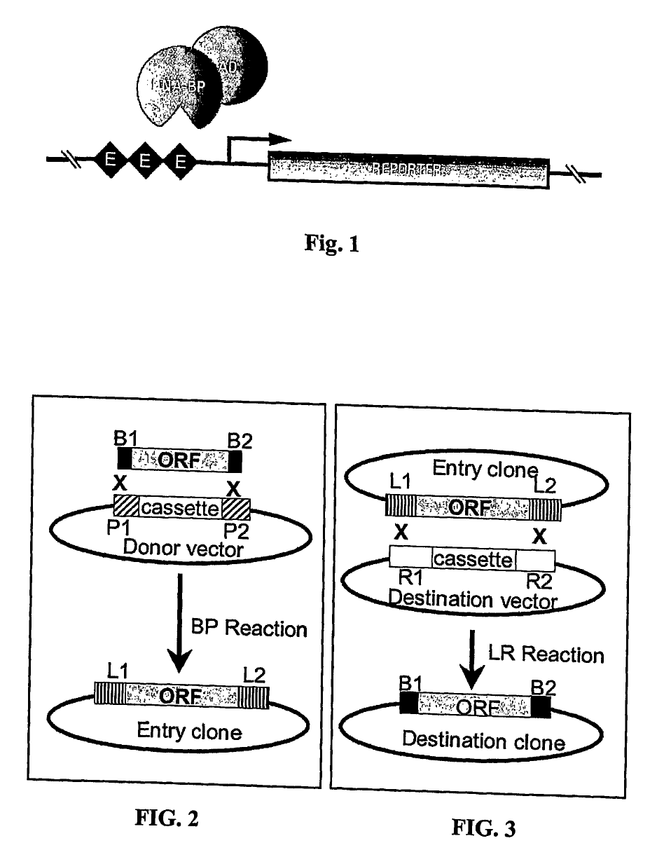

As shown in FIG. 1, in standard one-hybrid assays, multiple tandem copies of the bait element (shown as “E” in FIG. 1) are inserted upstream of a reporter gene creating a bait reporter construct. The figure shows how a DNA-binding protein (“DNA-BP”), and an associated activation domain (“AD”) can bind to a bait element to induce expression of the reporter gene. Theoretically, any bait element can be used to bind to (or “trap”) a protein having a binding domain specific for that element using these standard methods, as long as multiple copies of relatively short DNA fragments (e.g., up to about 25 to 30 bp) are used.

In the new, improved one-hybrid system described herein, one does not need to use multiple copies of a bait element and the bait element can be up to 2500 bp in length (e.g., from 50 to 2000, 250 to 1500, or 750 to 1000 bp), and the sequence of the entire bait element does not need to be known (just enough to prepare PCR primers to amplify bait element of interest). Instead, single copies of promoters, intergenic regions, introns, or large sections (up to about 2.5 kb) upstream of a predicted start codon are inserted into a reporter construct to create bait reporter constructs. Surprisingly, these single (and potentially large) bait elements in bait reporter constructs provide a sufficient signal, without excessive background “noise” and without the need for multiple tandem copies. In some embodiments, the bait element is cloned into a vector using the GATEWAY® cloning system or a modified version of the GATEWAY® cloning system.

To screen a library for a cDNA encoding a DNA-binding protein that interacts with a promoter sequence, cells (e.g., yeast) containing one or more bait-reporter constructs are transformed with an activation domain fusion library, and then the transformants are plated on a selective medium (e.g., as described in Fields et al., Nature, 340:245-247, 1998). If the activation domain fusion protein associates with the bait element, a reporter gene is expressed. As one example, expression of a HIS3 reporter gene allows colony growth on minimal medium lacking histidine and containing varying concentrations of 3-aminotriazole (3AT).

Methods for Making Vectors that Comprise a One-Hybrid Bait Reporter Construct

In general, the new one-hybrid bait reporter constructs are prepared by cloning a bait element upstream of a selected reporter gene to form one-hybrid bait reporter construct.

Although standard restriction and cloning methods can be used, one method of creating vectors containing a bait reporter construct from PCR products of the desired bait element, e.g., promoter PCR products, is to use a lambda recombination method, e.g., the GATEWAY® (Invitrogen, Carlsbad, Calif.) method. The general lambda recombination technology is based upon the in vitro utilization of both cis-acting recombination sites and trans acting recombinase enzymes of lambda (λ) integration. The wild-type lambda recombination involves recombination of the attP site of the phage DNA with the attB site on the bacterial genome. The integration reaction is called the “BP reaction.” After integration, the phage DNA is flanked by an attL site and an attr site. This integration is reversible and the reverse reaction is called the “LR reaction.”

In some embodiments, GATEWAY® methodology (Invitrogen, Carlsbad, Calif.) is used. In this method, the wild-type lambda recombination sites are modified into artificial sites, e.g., B1 and B2 for attB, P1 and P2 for attP. In each case, the duplications are created by mutations that create two different sites so that only the corresponding sites recombine with each other. In other words, B1 can recombine only with P1 and will result in L1 and R1 sites. Similarly, B2 recombines only with P2 to yield L2 and R2 sites. Likewise, L1 recombines only with R1 and results in D1 and P1 sites. Similarly, L2 recombines only with R2 to yield B2 and P2 sites. Thus, any DNA fragment flanked by B1 at its 5′ end and B2 at its 3′ end can be cloned uni-directionally into a vector (called the “donor vector”) containing P1 and P2 using the BP reaction (FIG. 2). This results in a PCR product cloned into a vector (called the “entry clone”) with the DNA fragment flanked by attL sites. The entry clone can now be used to clone the DNA fragment into any vector (called the “destination vector”) with compatible attR sites using the LR reaction (FIG. 3). The GATEWAY® Technology system is available commercially from Invitrogen (see, e.g., U.S. Pat. No. 5,888,732 and the GATEWAY® Technology manual Invitrogen, 2002)).

The selection of recombinant molecules originating from these recombination reactions can be enhanced by the inclusion of positive selection markers, e.g., an antibiotic resistance gene such as the kanamycin resistance gene (KanR), and negative selection markers, such as ccdB, in the donor vector. The ccdB-encoded protein interferes with the rejoining step of DNA gyrase, causing the chromosome of strains such as DH5I to be cut into pieces. This cell killing mechanism is encoded by the F plasmid in the wild (plasmids containing ccdb can be propagated in a gyrase mutant, gyrA462). In one embodiment, the donor vector includes a negative selection marker placed between the modified attP sites of the donor vector. In this case, an in vitro double recombination event replaces the toxic gene (e.g., ccdB) with the bait element, thereby allowing growth of transformed DH5α cells. Where the antibiotic resistance gene (e.g., KanR) is included in the donor vector, the selectivity is such that 99% of resistant colonies contain the desired clone. ccdB-containing recombinant linear molecules are also produced, but are eliminated upon transformation, since they cannot propagate in E. coli cells.

To insert a bait element into a reporter vector using the GATEWAY® system, the following general two step procedure can be used. First, the bait element of interest is produced by PCR using bait element specific primers synthesized with modified lambda recombination sites, e.g., modified lambda attB recombination sites such as attBl and attB4 sequences, at their 5′ ends. The PCR product including the modified attb sites flanking the bait element can now be cloned into a vector (the donor vector) that has two lambda recombination sites that match the modified lambda recombination sites in the bait element PCR product, e.g., a attp1 site (to match attB1) and a attP4 site (to match attB4), using a standard lambda recombination reaction, e.g., the BP reaction. The RP reaction creates an entry clone. Second, the entry clone vector containing the bait element is cloned into a reporter vector (the destination vector) using the LR reaction. (GATEWAY Technology Manual, No. 25-0522, Catalog nos. 12535-019 and 12535-027 (Invitrogen, Carlsbad, Calif.; 2003); Walhout et al., Methods in Enzymology 328:575-592, 2000).

In some embodiments, this two step procedure can be accomplished in one step using MultiSite GATEWAY® Technology, No. 25-0541, Catalog No. 12537-023 (Invitrogen, Carlsbad, Calif.; 2003).

The method of making an appropriate reporter vector or destination clone is known to those skilled in the art. In some embodiments, a cassette containing the ccdB gene and flanking lambda recombination sites (the GATEWAY® vector conversion cassette) is blunt-end ligated into a vector containing a reporter gene.

Integration of Vectors containing Bait-Reporter Constructs into Yeast Cells

Any yeast cell with appropriate genetic features (i.e., the appropriate genetic mutations to select for integration and transformation and reporter gene functionality) can be used in a one-hybrid screen. The yeast one-hybrid strain YM4271 (BD Biosciences, Palo Alto, Calif.) is one such yeast strain. To integrate bait-reporter constructs into the YM4271 genome, vectors containing bait-reporter constructs are linearized. Vectors containing HIS3 bait-reporter constructs can be linearized with Afill or XhoI, and then integrated at the his3-200 mutant locus by selecting integrants on Sc-His media. To generate double yeast integrants, vectors containing LacZ bait-reporter constructs can be linearized, e.g., with NcoI or ApaI, and integrated at the ura3-52 locus of HIS3-bait-reporter-containing strains. Careful selection of restriction enzymes is necessary to ensure that these restriction enzymes do not cut in the bait sequence. In general, for double integrants, the two bait elements of the two bait-reporter constructs are the same. Double integrants can be selected on Sc-His-Ura media. Transformation protocols are known in the art (Walhout et al, Methods, 24:297-306, 2001).

Self-Activation Control Experiments

Some of the endogenous transcription factors in yeast may bind to and activate the integrated bait-reporter constructs without the presence of an activation domain fusion protein, and thus some background expression may occur. As observed in the two-hybrid system for protein-protein interactions, it is possible that certain bait element/protein interactions will not be detectable in the new methods described herein if the bait elements (e.g., promoters) are self-activating. Because it is difficult to predict if a specific bait element is self-activating, a self-activation test can be conducted, e.g., as described herein. For each bait element, a colony that exhibits minimal self-activation with reporters should be chosen for use in subsequent yeast one-hybrid experiments.

Activation Domain Fusion Proteins

To generate activation domain-cDNA fusion proteins, a prey (e.g., a cDNA from a cDNA library) is cloned in frame with a heterologous activation domain. In some embodiments, the activation domain is from the yeast Gal4 transcription factor (e.g., ammo acids 768-881 of GAL4). In some embodiments, the activation domain of VP16 can be used.

Libraries of expression activation domain-cDNA fusion proteins are commercially available. In addition, methods of making libraries that express activation domain cDNA fusion proteins are known to those skilled in the art. The AD-worm cDNA library described in Walhout et al., Methods Enzymol., 328:575-92,2000, is an example of a suitable library. In addition, specific libraries containing only full-length transcription factor encoding open reading frames or open reading frames that encode transcription factor DNA binding domains can be used.

Methods of Detecting Activation Domain Fusion Protein/Bait Element Associations

The yeast one-hybrid (YlH) system (Li and Herskowitz, Science 262(5141):1870-4 (1993)) allows the identification of proteins that can bind to DNA elements of interest, including cis-regulatory elements, origins of DNA replication and telomeres (Lehming et al., Nature 371(6493):175-9 (1994); Li and Herskowitz (1993), supra; Kim et al., J. Biol. Chem. 278: 28038-28044). Briefly, the bait element is cloned upstream of a reporter gene (e.g., HIS3 or LacZ) and integrated into the yeast genome. Subsequently, a prey (e.g. a cDNA from a CDNA library) is transformed into the DNA bait::reporter yeast strains. If the prey contains a DNA binding domain that can interact with the DNA bait, reporter gene expression is activated via the fused activation domain. Although many predicted regulatory transcription factors contain an intrinsic activation domain, several transcription factors actually repress transcription and do not contain an activation domain. In addition, DNA binding proteins that do not function in transcription (e.g., replication and DNA repair proteins) do not contain an activation domain. To enable the identification of a wide variety of DNA binding proteins, the strong, heterologous Gal4 activation domain is added to the prey proteins.

The basic steps of a standard yeast one-hybrid system are described in the CLONTECH® MATCHMAKER® One-Hybrid System User Manual (PT 1031-1), Catalog No. K1603-1 (1997, Clontech Laboratories, Inc.).

In general, to screen a library for a cDNA encoding a DNA-binding protein that interacts with a particular bait element such as a promoter, yeast containing one or more bait-reporter constructs are transformed with an activation domain fusion library, and the transformants are plated on a selective medium at an appropriate dilution to generate a masterplate. After incubating the plates for two days at 30° C., colonies are replica-plated onto different plates to test the activation of different bait-reporter constructs.

For example, to evaluate the activation of the HIS3 bait-reporter construct, the yeast cells are grown on plates lacking histidine with increasing concentrations of 3AT (20-100 mM). To test the activation of the LacZ bait-reporter construct, the yeast cells are grown on YEPD plates for β-Gal assays. If the AD fusion protein interacts with the bait element, the HIS3 reporter gene is expressed, allowing colony growth on minimal medium lacking histidine and containing 3AT. 3AT is a competitive inhibitor of the His3 enzyme and, thus, higher amounts of His3 need to be expressed to confer growth in the presence of this compound. This provides a means to increase selectivity and reduce false positives that may be identified when background levels of His3 expression are relatively high. If two bait-reporter constructs are used, additional assays can be performed to confrm the interaction and eliminate false positives. For example, if both HIS3 and LacZ bait-reporter constructs are used, a beta-galactosidase assay can be performed to confirm the interaction and eliminate false positives. 3AT+, LacZ+ positive clones are candidates for expressing activation domain DNA binding protein fusions that specifically bind to the bait element of interest. Protocols for identifying the open reading frame of positive clones are known to those skilled in the art (Sambrook et al., eds., Molecular Cloning: A Laboratory Manual. 2nd ed., Cold Spring Harbor Laboratory, Cold Spring Harbor Laboratory Press, Cold Spring Harbor, N.Y., 1989; Walhout et al., Methods 24: 297-306, 2001).

Use of the One-Hybrid System to Develop a “DNA-Protein Interaction” Map

The regulation of transcription in response to biological signals plays a central role in many biological processes such as proliferation, inhibition of apoptosis, differentiation, and cell fate-determination, which are important in both healthy cells and tumor cells. Several regulatory transcription factors, including c-Myc, pRB, the Wilms' tumor protein and the estrogen receptor, have been described as playing a direct role in these processes. Aberrant forms of regulatory transcription factors can be generated by chromosomal mutations, leading to altered functional activities and tumorigenesis. One of the best known examples involves the p53 transcription factor that regulates apoptosis and is mutated in many human cancers. In addition, the function of transcription factors can be dramatically altered because of chromosomal translocations. For example, translocations of the c-myc oncogene have been described in B-cell lymphomas.

The new yeast one-hybrid methods described herein can be used to obtain a detailed picture of the expression of regulatory transcription factors and their targets under physiological and disease conditions in the context of an intact organism. For example, the new one-hybrid screen can be performed bi-directionally: 1) to identify regulatory transcription factors that can interact with a promoter of interest, and 2) to identify promoters that are regulated by a regulatory transcription factor of interest.

After performing the one-hybrid screens described herein, the subsequent procedures are similar to that of known, large-scale, semi-automated two-hybrid screens. Positive colonies are tested for different one-hybrid phenotypes, and can be examined for “promoter self-activation.” Subsequently, the identity of the open reading frame is determined by yeast colony PCR and sequencing. The relevance of potential promoter regulators is examined by comparing the expression patterns of regulatory transcription factors and their targets. In other words, when a transcription factor serves to activate a promoter, the expression pattern of it should overlap, at least partially, with its target. However, if a transcription factor represses a promoter, the expression patterns should not partially overlap. To make this distinction, transcription factor function can be addressed using the corresponding native open reading frame in yeast one-hybrid assays.

In addition, whether the transcription factor up-regulates or down-regulates a promoter can be examined using promoter-fluorescent protein fusions in mammalian cells by transfection experiments. A number of fluorescent proteins suitable for use are known in the art and are commercially available, including, but not limited to, luciferase, green fluorescent protein, yellow fluorescent protein, red fluorescent protein, blue fluorescent protein, or a fluorescent variant thereof. These methods are known to those skilled in the art.

A yeast one hybrid experiment can be performed in a matrix fashion. As described in Walhout et al., Methods 24: 297-306, 2001, matrix experiments can be done by mating using strains of opposite mating types that carry either the bait-reporter construct or the individual activation domain transcription factor fusion proteins. One mating type is streaked out on a plate in a horizontal fashion (each horizontal row containing a different bait-reporter construct or different fusion protein) and the other is streaked out in a vertical fashion (each column contain a different bait reporter construct or different fusion protein). The two strains are replicate plated onto rich medium to allow diploid formation and then replicate plated onto selective medium to select for diploid cells. These diploid cells will contain both the bait reporter construct and a fusion protein and can be tested for bait element—fusion protein associations, e.g., binding, as described herein.

### EXAMPLES

The invention is further described in the following examples, which do not limit the scope of the invention described in the claims.

### Example 1

### Generation of Vectors Containing a Bait Reporter Construct

To create a vector containing a DNAbait::reporter construct, two reporter genes, HIS3 and LacZ, were selected. The use of two reporters simultaneously allows the assessment of putative protein-DNA interactions with both reporters in the same cell. This greatly reduces the number of false positives identified. New vectors that contain the reporter were generated. These vectors contain R4 and L1 recombination sites that flank the Gateway cloning cassette R4-L1::reporter). The Y1H reporter genes are cloned downstream of the Gateway cassette to create R4-L1::reporter::B2 plasmids.

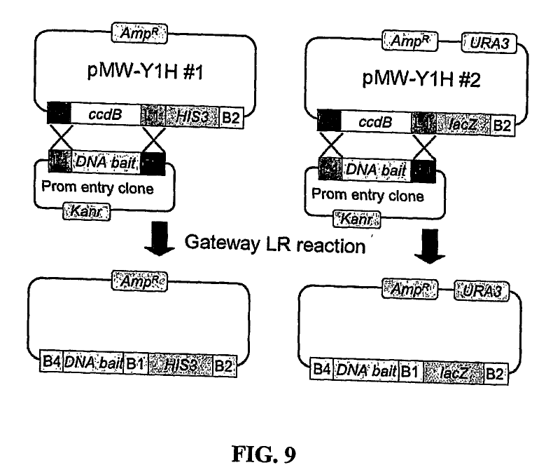

Existing DNAbait: :reporter plasmids were used to generate the appropriate vectors. Two promoter-reporter clones were used to generate vectors that contain both a R4-L1::reporter and either HIS3 or LacZ. To create pMW-YlH#1, containing a HIS3 reporter (FIG. 9), a Pmec-7::HIS3 Destination clone was used in a BP cloning reaction together with pDONR-P4-PlR to generate 2 new products: a vector containing the R4-L1::HIS3::B2 fusion and a Pmec-7 Entry clone. Cloning reactions were transformed into DB3.1 cells that are tolerant to the Gateway cassette, and bacteria containing the desired vector were retrieved by selection on media containing ampicillin and chloramphenicol.

pDEST-YlH#2, containing a LacZ reporter (FIG. 9) was generated in similar fashion, except that a Phlh-8::lacZ Destination clone was used.

### Example 2

### Lambda Recombination Sites Do Not Interfere with Reiporter Activation

To determine whether the lambda recombination site inserted between the bait element and the reporter gene in bait-reporter constructs would interfere with the activation of reporter gene, the following experiment was done.

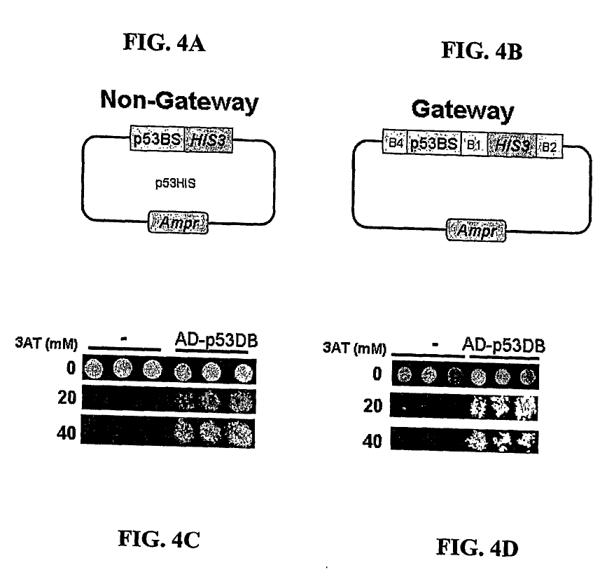

A bait-reporter construct containing 6 copies of the murine consensus p53 binding site (p53BS) upstream of the HIS3 reporter gene was used with Gal4 activation domain alone (−) and with Gal4 activation domain fused to the binding domain of p53 (FIGS. 4A and 4C). In a parallel experiment, the same bait-reporter construct was used except that a lambda recombination site (B1) was inserted between the p53 binding sites and the HIS3 reporter gene. In this parallel experiment, the bait-reporter construct was tested with Gal4 activation domain alone (−) and with Gal4 activation domain fused to the binding domain of p53 (FIGS. 4B and 4D). Cells were spotted on Sc-His,-Ura media with 0, 20, or 40 mM 3AT as indicated. As shown in FIGS. 4B and 4D, the lambda recombination sites do not make a difference in the activation of the HIS3 gene.

### Example 3

### Bait Reporter Construct Self-Activation Test

In the experiments described in this example, self-activation by two C. elegans promoters was tested.

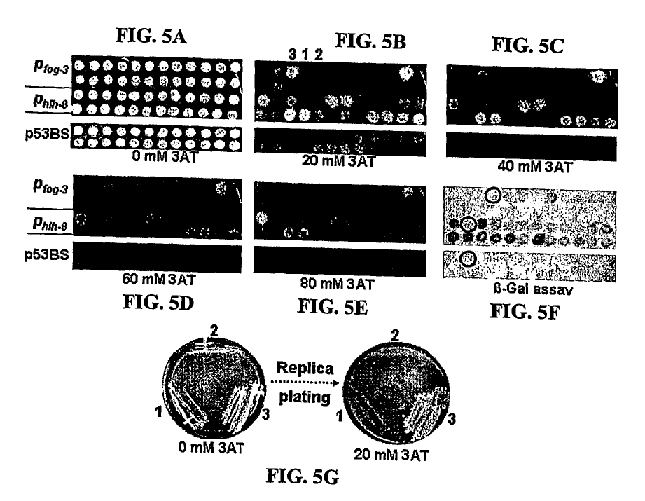

The promoter of the C. elegans fog-3 gene was cloned by lambda recombination into a HIS3 bait-reporter construct and a LacZ bait-reporter construct using the Multisite GATEWAYS system (Invitrogen, Carlsbad). These two constructs were sequentially integrated into the genome of YM4271 yeast. Similarly, the promoter of the C. elegans hlh-8 gene was cloned into a HIS3 bait-reporter construct and a LacZ bait-reporter construct. These two constructs were sequentially integrated into the genome of YM4271 yeast. Twenty-four HIS3/LacZ double integrant colonies were picked and spotted either onto Sc-His-Ura media plus increasing amounts of 3AT (0, 20, 40, 60 and 80 mM) to determine self-activation of the HIS3 reporter, and onto YEPD plates containing a nitrocellulose filter to analyze lacZ bait-reporter construct self-activation using a β-Gal assay. (FIGS. 5A-5F; circles in 5A indicate colonies that were selected for downstream further experiments; a bait-reporter constructs containing multiple p53 binding sites was used as control). Growth on plates containing 3AT or dark circle on the #1-Gal assay represent colonies that were self-activating. For each bait element, a colony that exhibits minimal self-activation with both reporters should be chosen for use in subsequent yeast one-hybrid experiments.

To determine if individual colonies exhibit phenotypic variations after clonal expansion, three fog-3 promoter colonies, varying in their extent of self-activation (low=1, moderate=2, and high=3) were picked and restreaked on Sc-His-Ura media after which they were replica-plated onto selective 20 mM 3AT-containing media. (FIG. 5G). Phenotypic variations in propagated colonies were not observed, indicating that the level of self-activation was stable, making yeast one-hybrid experiments possible.

### Example 4

### Activation of the HIS3 Reorter by Certain Transcription Factor-Bait Element Associations

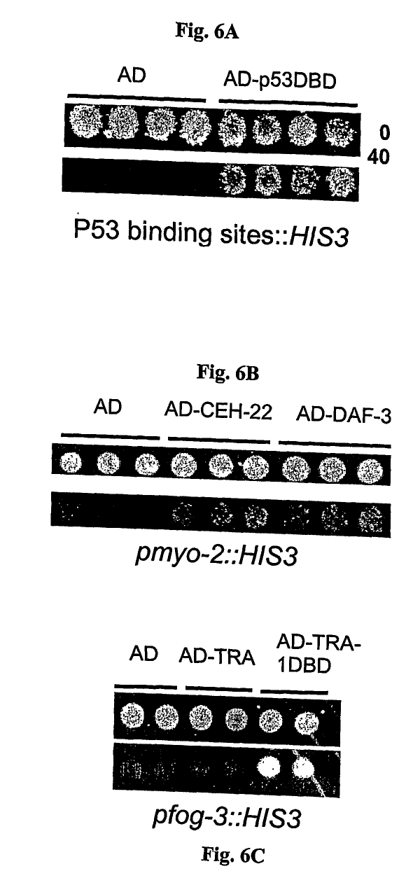

Various experiments were performed to show how the P53BS, myo-2, mab-3, and fog-3 promoters interact with various known transcription factors under selective conditions (bottom row in FIGS. 6A-C, with 40 or 60 mM 3AT as indicated). The top row demonstrates that under permissive conditions, all colonies were able to grow (e.g. also in the absence of a prey protein-bait DNA interaction), indicating that the yeast cells were present.

FIG. 6A shows the results of the first experiment. Briefly, a bait-reporter construct containing 6 copies of the murine consensus p53 binding site upstream of the HIS3 reporter gene was used with Gal4 activation domain alone (AD) and with Gal4 activation domain fused to the binding domain of p53 (AD-p53DBD). There was no growth of colonies in the presence of the AD alone, but there was clear growth in the presence of AD-p53DBD protein (at 40 mM 3AT).

FIG. 6B shows the results when promoter mab-3 was combined with the HIS3 promoter and grown in the presence of various proteins. There was minimal to no growth with just AD, slight growth in the presence of AD-TRA-1 (a transcription factor known to bind to the mab-3 promoter), and good growth in the presence of AD-TRA-1DBD (the DNA binding domain of TRA-1; at 40 mM 3AT).

FIG. 6C shows the results whenfog-3 was combined with HIS3 and grown in the presence of AD alone (very little, if any, growth, thus not much self-activation), AD-TRA-1 (a transcription factor known to bind to the fog-3 promoter; minimal growth), and AD-TRA-1DBD (very strong growth a 40 mM 3 AT).

These results demonstrate that the HIS3 reporter was activated when a transcription factor fusion protein known to bind a particular bait element was expressed in yeast cells containing that particular bait element. In other words, expression of a transcription factor known to bind to bait element X in a yeast cell containing bait element X corresponds to activation of the HIS3 reporter gene.

### Example 5

### Activation of the LacZ Reporter by Certain Transcription Factor-Bait Element Associations

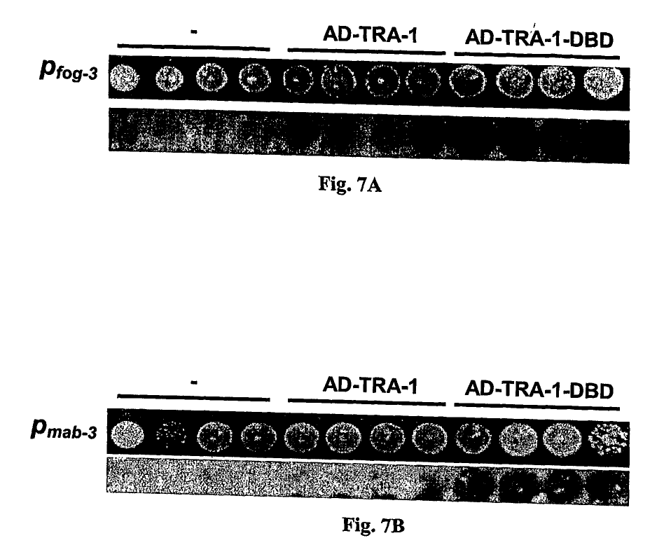

To test the promoters of myo-2, fog-3, and mab-3 were cloned upstream of the LacZ gene and tested for their interaction with known transcription factors under permissive conditions (top row in FIGS. 7A-B, in which all colonies grew) and beta-galactosidase assay conditions (bottom row in FIGS. 7A-B, with dark gray color indicating the blue, positive colonies).

FIG. 7A shows the results when the fog-3 promoter was combined with LacZ reporter and grown in the presence of no protein (no blue color), AD-TRA-1 (a transcription factor known to bind to thefog-3 promoter)(minimal blue color), and AD-TRA-1DBD (strong blue color).

FIG. 7B shows the results when the mab-3 promoter was combined with the LacZ promoter and grown in the presence of various proteins. There was minimal to no blue color with no protein, slight blue color in the presence of AD-TRA-1 (a transcription factor known to bind the mab-3 promoter), and a solid blue color in the presence of AD-TRA-1DBD.

### Example 6

### Detecting Protein-Promoter Interactions Using an AD-TF Mini-Library

To increase the likelihood of retrieving transcription factors that are present in low proportions in an activation domain cDNA fusion library, a C. elegans activation domain transcription factor fusion mini-library (“AD-TF mini-library”) was generated. To generate the AD-TF mini-library, a list of 1,010 transcription factor coding genes were identified in the worfdb database which contains information about the ORFeome cloning project (Vaglio et al., Nucleic Acids Research 31:237-240, 2003). A total of 634 clones encoding transcription factors (˜63%) were available in the ORFeome; the remaining 37% were not yet available because they could not be obtained as full length open reading frames from the cDNA libraries (Reboul et al., Nature Genetics 34: 35-41). The 634 clones were picked and DNA was purified in an automated manner using a Biorobot 8000 (Qiagen). Subsequently, all 634 clones were sub-cloned by high-throughp ut Gateway LR cloning into pDESTAD (based on pDEST22 (Invitrogen, Carlsbad, Calif.)) to generate activation domain transcription factor fusions. These fusions can be used both in yeast one hybrid and yeast two hybrid experiments. Clones were analyzed by PCR to examine if they contain an insert of the expected size. The activation domain transcription factor fusion mini-library (“AD-TF mini-library”) was generated by mixing the glycerol stocks of individual activation domain transcription factor fusion clones in equal amounts and purifying mixed plasmid DNA from the pooled clones using CsCl₂-based purification.

The AD-TF mini-library contains the full-length open reading frame of ˜63% of all predicted C. elegans transcription factors in the correct orientation and frame and in roughly equimolar amounts. Because this mini-library has a highly reduced complexity compared to the AD-cDNA library, fewer colonies have to be screened. Each promoter bait element was screened using the AD-cDNA library and the AD-TF mini-library (Table 1). Each double positive was retested by PCR/gap-repair, described previously in Walhout et at., Methods 24:297-306,2001. For gap repair based phenotypic retesting, yeast colony PCR was performed as Gap repair was performed as described in Walhout et at., 2001, supra.

TABLE 1
Overview of yeast one-hybrid screen results

| | | | | | |
|---|---|---|---|---|---|
| | Total # of | | | | |
| DNA | colonies | Picked | Double | Retest by | Relevant |
| Bait | screened | colonies | positives | Gap-repair | hits |

AD-cDNA library

| | | | | | |
|---|---|---|---|---|---|
| P_{fog-3} | 1.2 × 10⁵ | 60 | 4 | — | 0 |
| P_{mab-3} | 1.8 × 10⁵ | 48 | 4 | — | 1 |
| P_{myo-2} | 1.8 × 10⁵ | 67 | —* | 7 | 4 |
| Pₕₗₕ₋₈ | 1.1 × 10⁶ | 15 | —* | 3 | 3 |

AD-TF mini-library

| | | | | | |
|---|---|---|---|---|---|
| P_{fog-3} | 1.4 × 10⁶ | 45 | 22 | 5 | 5 |
| P_{mab-3} | 6.2 × 10⁵ | 48 | 0 | — | — |
| P_{myo-2} | 1.2 × 10⁶ | 41 | —* | 2 | 2 |
| Pₕₗₕ₋₈ | 5.6 × 10⁵ | 44 | —* | 0 | — |

*The β-Gal test could not be performed for these promoters because of high lacZ reporter self-activation.

Although no positives were found for the fog-3 promoter with the AD-cDNA library, 5 double positives were found with the AD-TF mini-library (Table 1). The first fog-3 promoter interactor, B0261.1, is a novel protein with a predicted Myb-like DNA binding domain. The second interactor, ODR-7, is an olfactory-specific member of the nuclear hormone receptor superfamily (Sengupta et al., Cell 79:971-980, 1994). The remaining interactors correspond to CEH-17, a homeodomain-containing transcription factor that has been implicated in axonal development (Pujol et al., Development 127: 3361-3371, 2000).

Yeast one-hybrid screens with the AD-TF mini-library yielded two additional putative myo-2 promoter interactors. F22D6.2 is a zinc finger-containing protein with unassigned function. NHR-123 is a novel nuclear hormone receptor that has not been studied in C. elegans, but exhibits significant homology with the human vitamin D3 receptor. Interestingly, human vitamin D3 receptor regulates the expression of several genes involved in muscle development, including myosin heavy chain genes (Endo et al., Endocrinology 144:5138-5144,2003). Since myo-2 encodes a myosin heavy chain, the NHR-123 association with the myo-2 promoter may constitute an “interolog,” i.e., an evolutionarily conserved interaction (Walhout et al., Science 287:116-122, 2000).

These results demonstrate the ability of the AD-TF mini-library to find protein-DNA associations that would not have been easily found with a convention cDNA activation domain library. These results also generally demonstrate the ability of the methods described herein to identify novel protein-DNA associations (e.g., binding).

### Example 7

### Validation of Yeast One-Hybrid Interactions

To validate the yeast one hybrid based protein DNA interactions, we performed transient co-transfections and luciferase reporter assays were performed in mouse 3T3 cells, focusing on interactions detected with the fog-3 promoter, as all the corresponding transcription factor encoding open reading frames are available as entry clones in the C. elegans ORFeome (Reboul et al., Nature Genetics 34:35-41, 2003). The fog-3 promoter was cloned into a GATEWAY® compatible luciferase reporter vector that contains a minimal SV40 promoter using standard methods. A cassette containing the ccdB gene and flanking lambda recombination sites (the GATEWAY vector conversion cassette) was blunt-end ligated into a vector containing the luciferase reporter vector and SV40 promoter. The open reading frames encoding putative fog-3 promoter interactors (including B0261.1, TRA-1, and TRA-1DB) were sub-cloned into an N-terminal GST tag-containing vector that allows high-level protein expression in mammalian cells. Each transfection was performed in triplicate and independently repeated 3 times.

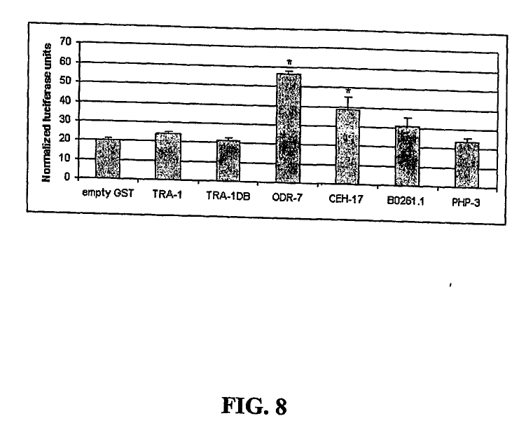

The results of a representative experiment are shown in FIG. 8. Both ODR-7 and CEH-17 significantly induced luciferase expression compared to negative controls, confirming that these transcription factors can bind to the fog-3 promoter. No significant inductions were observed forB0261.1, TRA-1, and TRA-1DB. The lack of induction by TRA-1 may be because TRA-1 functions as a repressor in C. elegans (Chen et al., Development 127:3119-3129, 2000). It is important to note that no heterologous transcription activation domain was added to the transcription factor for this assay. Thus, the data obtained suggest that both ODR-7 and CEH-17 may function as transcriptional activators in vivo.

### Other Embodiments

A number of embodiments of the invention have been described. Nevertheless, it is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a protein that binds to a bait element, the method comprising:
obtaining a cell whose genome comprises one or more integrated bait-reporter constructs, wherein each of the one or more bait-reporter constructs comprises
(a) a single copy of a bait element flanked by lambda recombination sites, wherein the bait element comprises at least 250 base pairs; and
(b) a reporter gene;
transforming the cell with an expression vector encoding a fusion protein comprising an activation domain; and
assessing activation of the reporter gene,
wherein activation of the reporter gene indicates that the activation domain fusion protein has bound to the bait element.

2. The method of claim 1, wherein the cell is a yeast cell.

3. The method of claim 2, wherein the yeast cell is a YM4271 cell.

4. The method of claim 2, wherein the reporter gene is HIS3.

5. The method of claim 2, wherein the reporter gene is LacZ.

6. The method of claim 1, wherein two reporter genes are used.

7. The method of claim 6, wherein the cell is a yeast cell and the reporter genes are HIS3 and LacZ.

8. The method of claim 1, wherein the cell is a mammalian cell.

9. The method of claim 8, wherein the reporter gene is a fluorescent gene.

10. The method of claim 9, wherein the fluorescent gene is selected from the group consisting of luciferase, green fluorescent protein, yellow fluorescent protein, red fluorescent protein, blue fluorescent protein, or a fluorescent variant of any of said proteins.

11. The method of claim 1, wherein the bait element comprises more than 500 base pairs.

12. The method of claim 1, wherein the fusion protein is an activation domain-transcription factor fusion protein.

13. The method of claim 1, wherein assessing activation comprises plating the yeast cell on nutrient deficient plates under growth conditions, and observing any growth of yeast cells.

14. The method of claim 1, wherein assessing activation comprises determining the color of the yeast cells.

15. The method of claim 1, further comprising
selecting a cell in which a reporter gene is activated; and
isolating the cDNA encoding an activation domain fusion protein that is bound to the bait element from the cell.

16. The method of claim 15, further comprising determining the sequence of the isolated cDNA.

17. The method of claim 1, further comprising contacting the cell with a test compound, and evaluating the effect of the test compound on binding of the bait element to the activation domain fusion protein.
